(19) United States
(12) Patent Application Publication
(10) Pub. No.: US 2003/0003139 A1
(43) Pub. Date: Jan. 2, 2003

(54) Transdermal system that contains a new highly potent gestagen

(75) Inventors: Ralph Lipp, Berlin (DE); Clemens Gunther, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 10/022,834

(22) Filed: Dec. 20, 2001

(30) Foreign Application Priority Data
Dec. 21, 2000 (EP) 00250449.6

(51) Int. Cl. A61K 31/57; A61K 9/70

(52) U.S. Cl. 424/449; 514/178

(57) ABSTRACT

Matrix-transdermal system that contains (21S)-21-hydroxy-21-methyl-14,17-ethano-19-norpregna-4,9,15-triene-3,20-dione that comprises a polyacrylate adhesive.

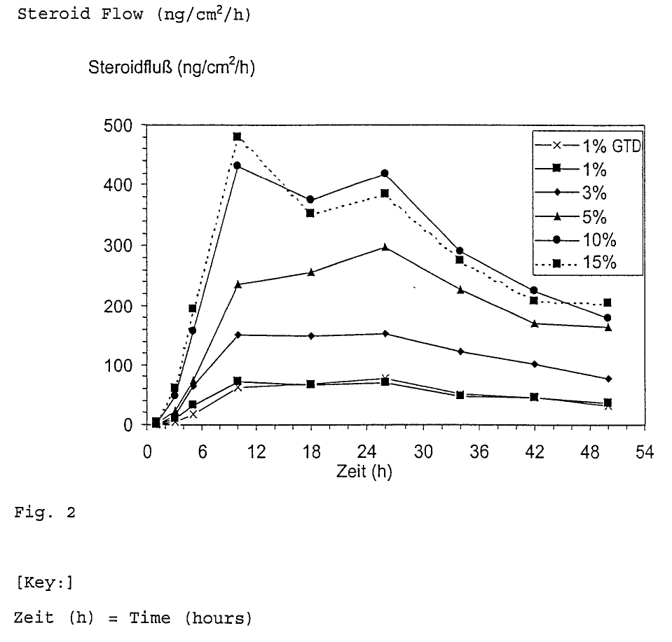

The invention relates to a transdermal system (TDS) of highly potent gestagen(s), especially of (21S)-21-hydroxy-21-methyl-14,17-ethano-19-norpregna-4,9,15-triene-3,20-dione, referred to below as hydroxytrienedione (FIG. 1).

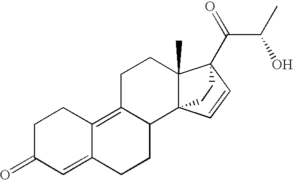

FIG. 1: Structural formula of hydroxytrienedione

Transdermal systems are a special form of transdermal formulations.

For the purpose of the application, the term “transdermal system” is used in a narrow definition for all transdermal patch formulations. Usually, transdermal systems are subdivided into matrix-transdermal systems and membrane-transdermal systems.

In the simplest case, matrix-transdermal systems consist of three layers that are arranged parallel over one another, namely a back layer, a matrix and a peel-off layer. The latter, which usually consists of plastic film or coated paper, is removed before the transdermal system is applied to the skin. The matrix contains the active ingredient that is to be administered and usually simultaneously has adhesive properties. Should the matrix not be adhesive enough to adhere reliably to the skin area on which the transdermal system is to be administered, an adhesive layer can still be provided between matrix and peel-off layer.

In the simplest case, membrane-transdermal systems consist of four layers, namely a back layer, a reservoir, a membrane and a peel-off layer. The reservoir, which can contain active ingredients and adjuvants, is usually completely surrounded by back layer and membrane. The components of the reservoir can be released through the membrane. Since a number of membranes are not sufficiently adhesive to adhere to the skin area on which the transdermal system is to be administered, an adhesive layer is generally provided between membrane and peel-off layer (at least in the edge area). It is known that gestagens can be administered transdermally. The gestagens previously used in formulations for transdermal systems generally have relatively low solubilities in the matrices used, however, e.g., gestodene or levonorgestrel about 1%. A crystal-free transdermal system can be produced with these gestagens only if the content of gestagen in the matrix does not significantly exceed the saturation concentration.

A high content of gestagen in the matrix is often desired to achieve sufficient transdermal skin flows.

Other gestagens have a higher solubility, such as, e.g., norethistosterone (about 7%), but their gestagenic potency is comparatively low.

The object of the invention is therefore to make available a transdermal system with a high content of highly potent gestagens in dissolved form.

According to the invention, this object is achieved by a transdermal system with a content of the highly potent gestagen (21S)-21-hydroxy-21-methyl-14,17-ethano-19-norpregna-4,9,15-triene-3,20-dione (hydroxytrienedione).

In an embodiment, the invention provides that (21S)-21-hydroxy-21-methyl-14,17-ethano-19-norpregna-4,9,15-triene-3,20-dione is present in a matrix, especially an adhesive matrix.

As medically acceptable adhesives, for example, polyacrylate, silicone or polyisobutylene adhesives can be used. Moreover, polyurethanes, block-copolymers based on styrene and other organic polymers can be used, however.

Polyacrylate adhesives are preferred.

For the purpose of the patent, polyacrylate is the generic term for all polymers (homopolymers and copolymers) that contain acrylic acid or acrylic acid derivatives. Especially preferred are vinyl acetate-acrylate-copolymers and acrylate-vinylpyrrolidone-copolymers, and most preferred are 2-ethylhexylacrylate-hydroxyethylacrylate-copolymers (Gelva^{(R)}) as well as copolymers of the above-mentioned compounds with other substances, such as, for example, vinyl acetate and 2-ethylhexylacrylate-N-vinyl-2-pyrrolidone (TSR^{(R)}-adhesive of the Sekisui Company).

A content of 0.1 to 20% by weight of hydroxytrienedione ((21S)-21-hydroxy-21-methyl-14,17-ethano-19-norpregna-4,9,15-triene-3,20-dione), preferably 1 to 15% by weight, is provided in the matrix.

In an especially preferred embodiment, the transdermal formulation according to the invention contains crystallization inhibitors that are suitable as complexing agents to form, for example, solid solutions with active ingredient that increase the interfacial solubility for the active ingredient and reduce the tendency of the active ingredient to recrystallization after a process solvent is removed or the temperature is dropped. The addition of crystallization inhibitors makes it possible to undertake higher active ingredient loadings of the formulation without active ingredient crystals forming, which are available to an only very limited extent for mass transfer into the skin. As crystallization inhibitors, N-vinyllactam polymers, such as N-vinyl-1-aza-cycloheptan-2-one-homopolymers and N-vinyl-piperidin-2-one-homopolymers and especially polymers of vinylpyrrolidone, such as polyvidone (Kollidon^{(R)}) or co-polymers of vinylpyrrolidone with vinyl acetate (copovidones) are suitable. Especially preferred is a copovidone that consists of 6 parts vinylpyrrolidone and 4 parts vinyl acetate (Kollidon^{(R)} VA 64).

The content of crystallization inhibitor in the transdermal system according to the invention is 0.1 to 40%, preferably 2 to 20%.

The system according to the invention preferably has an additional content of at least one penetration intensifier.

As penetration intensifiers, the following can be used:

Monovalent or multivalent alcohols such as ethanol, 1,2-propanediol or benzyl alcohol; saturated or unsaturated fatty alcohols with 8 to 18 carbon atoms, such as lauryl alcohol or cetyl alcohol; hydrocarbons such as mineral oil; saturated and unsaturated fatty acids with 8 to 18 carbon atoms such as stearic acid or oleic acid; fatty acid esters with up to 24 carbon atoms or dicarboxylic acid diesters with up to 24 carbon atoms, such as methyl ester, ethyl ester, isopropyl ester, butyl ester, sec-butyl ester, isobutyl ester, tert-butyl ester or monoglyceric acid ester of acetic acid, caproic acid, lauric acid, myristic acid, stearic acid and palmitic acid, phosphatide derivatives, such as lecithin, terpenes, urea and its derivatives or ethers, such as dimethyl isosorbide and diethylene glycol monoethyl ether.

Especially preferred are lauryl alcohol, 1,2-propanediol, methyl ester and especially the isopropyl ester of myristic acid or oleic acid, diisopropyl adipate and diisopropyl sebacate, lauric acid and oleic acid, as well as mixtures thereof.

In addition, mixtures that consist of one or more penetration-intensifying agents can also be used for the transdermally active formulation according to the invention. Here, mixtures of up to four penetration intensifiers are used. The use of binary and ternary penetration intensifier mixtures is preferred. Most preferred is the use of binary penetration intensifier mixtures that consist of hydrophilic with lipophilic penetration intensifiers.

Examples are enhancer mixtures of fatty acid esters or fatty acids with short-chain, monovalent or divalent alcohols in a ratio of 1:10 to 10:1. A preferred mixture ratio is 3:1 to 1:3.

The content of penetration intensifiers or penetration intensifier mixtures in the transdermal system according to the invention is 0.5 to 40%, preferably 5 to 25%.

Moreover, stabilizers such as cyclodextrins, preferably β-cyclodextrin and derivatives thereof, can also be added to the matrix.

In addition, the invention is preferably characterized by an additional content of at least one estrogen.

As estrogens, the following can be used: estradiol, estriol, ethinylestradiol, their derivatives such as, for example, mono-esters and di-esters, such as estradiol-3,17β-diproprionate.

For the first time, a highly potent gestagen is made available, surprisingly enough, with (21S)-21-hydroxy-21-methyl-14,17-ethano-19-norpregna-4,9,15-triene-3,20-dione (hydroxytrienedione), and said gestagen has, in transdermal systems, especially matrix-transdermal systems based on polyacrylate adhesive, a surprisingly high solubility of up to about 20% by weight. Extraordinarily high transdermal flows can be achieved using such highly loaded systems.

The combination of high gestagenic potency, excellent matrix-loadability and skin penetration of the active ingredient makes it possible to make available efficient hormone replacement transdermal systems or birth control transdermal systems with use of hydroxytrienedione.

Of course, it is possible to include other adjuvants, such as the above-mentioned crystallization inhibitors and penetration intensifiers or solubilizers and/or solvents in the matrix-transdermal systems. Moreover, penetration enhancers can also be applied to increase the skin flows even before administration of the transdermal system to the corresponding skin parts.

The features of the invention that are disclosed in the description above as well as in the claims can be essential both individually and in any combination for the implementation of the invention in its various embodiments.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosure of all applications, patents and publications, cited above [or below], and of corresponding European Patent application No. 00250449.6, filed Dec. 21, 2000 is hereby incorporated by reference.

### EXAMPLES

### Example 1

### Production of Transdermal Systems with Hydroxytrienedione

Hydroxytrienedione is weighed in a beaker and with 25 g of a 30% solution of Kollidon^{(R)} VA 64 in 2-propanol. 10 ml of 2-propanol is added. The mixture that is obtained is stirred for 5 minutes. The mixture is fed into the adhesive solution (Gelva 7881) that is introduced. The following table indicates the adhesive dry mass that is contained in the introduced volume of the adhesive solution. Then, it is stirred for another 0.5 hour. After polarization-microscopic checking to ensure the absence of crystals, the mixture that is obtained is coated by means of a 300 μm doctor blade on a liner that consists of fluoropolymer-coated polyester (Scotchpak^{(R)} 1022 Release Liner). It is dried for 20 minutes at 70° C. and then laminated with a backing that consists of a co-laminate of PVC-PVDS with polyester (Saran-Hytrel^{(R)} backing). Round patches with a surface area of 10 cm² are punched from the three-layer laminate that is obtained and welded into a sealed bag that is made of aluminum compound foil.

| | | | Hydroxytriene- | | | |
|---|---|---|---|---|---|---|
| | Polyacrylate | | dione | | Kollidon^{(R)} | |
| | adhesive | | Concentration | | VA 64 Conc. in | |
| Formulation | (Gelva^{(R)} 7881) | | in TDS (m/m %) | | TDS (m/m %) | |
| 1 | 42 | g | 0.5 g | 1 | 7.5 g | 15 |
| 2 | 41 | g | 1.5 g | 3 | 7.5 g | 15 |
| 3 | 40 | g | 2.5 g | 5 | 7.5 g | 15 |
| 4 | 37.5 | g | 5.0 g | 10 | 7.5 g | 15 |
| 5 | 35 | g | 7.5 g | 15 | 7.5 g | 15 |

Gelva^{(R)} 7881 is a 50% polyacrylate adhesive solution in ethyl acetate (manufacturer: Solutia Company). In the above table, the dry weights are indicated as amounts weighed-in.

Scotchpak^{(R)} 1022 Release Liner is a product of the 3M Company, St. Paul, Minn., USA. Saran-Hytrel^{(R)} backing is a product the Bertek Company, St. Albans, Vt., USA.

### Example 2

### Penetration Studies

The results of the penetration studies with the corresponding five formulations with different contents of hydroxytrienedione are shown in the following tables.

The measurements were made with the following in-vitro-diffusion test. A tempered Franz-flow cell is divided into a donor compartment and an acceptor compartment by a 2 cm² piece of excised skin from nude mice, whereby the horny layer faces toward the donor. A 3% or 5% solution of hydroxypropyl-β-cyclodextrin in buffer is pumped with the aid of a pneumatic pump from a tempered storage container through the acceptor compartment and collected with the aid of a fraction collector in glass vials that are exchanged at specific time intervals.

The active ingredient-containing patches are bonded to the donor side.

The content of active ingredients is determined in the individual fractions by means of HPLC/UV or GC/FID.

The transdermal in-vitro skin flow is indicated in ng/cm²/h:

Formulation 1: 1% Hydroxytrienedione

| [h] | 1 | 2 | 3 | 4 | MW | SD | CV |
|---|---|---|---|---|---|---|---|
| 1 | 2 | 1 | 0 | 0 | 1 | 1 | 86.6% |
| 3 | 16 | 13 | 2 | 6 | 9 | 6 | 70.4% |
| 5 | 55 | 36 | 11 | 27 | 32 | 18 | 57.2% |
| 10 | 110 | 57 | 45 | 72 | 71 | 28 | 39.8% |
| 18 | 79 | 59 | 51 | 76 | 66 | 13 | 20.2% |
| 26 | 77 | 44 | 48 | 111 | 70 | 31 | 44.3% |
| 34 | 56 | 32 | 47 | 52 | 47 | 11 | 22.6% |
| 42 | 62 | 26 | 40 | 51 | 45 | 15 | 33.9% |
| 50 | 40 | 20 | 39 | 42 | 35 | 10 | 29.0% |

Formulation 2: 3% Hydroxytrienedione

| [h] | 1 | 2 | 3 | 4 | MW | SD | CV |
|---|---|---|---|---|---|---|---|
| 1 | 1 | 1 | 0 | 1 | 1 | 0 | 71.1% |
| 3 | 20 | 24 | 4 | 16 | 16 | 9 | 54.3% |
| 5 | 64 | 111 | 17 | 61 | 63 | 38 | 60.6% |
| 10 | 118 | 171 | 109 | 207 | 151 | 46 | 30.6% |
| 18 | 118 | 144 | 166 | 161 | 148 | 22 | 14.6% |
| 26 | 106 | 147 | 197 | 158 | 152 | 37 | 24.5% |
| 34 | 71 | 115 | 160 | 142 | 122 | 39 | 31.8% |
| 42 | 54 | 114 | 120 | 116 | 101 | 31 | 30.9% |
| 50 | 46 | 91 | 68 | 103 | 77 | 25 | 32.8% |

Formulation 3: 5% Hydroxytrienedione

| [h] | 1 | 2 | 3 | 4 | MW | SD | CV |
|---|---|---|---|---|---|---|---|
| 1 | 2 | 1 | 2 | 2 | 1 | 1 | 67.3% |
| 3 | 33 | 18 | 13 | 24 | 22 | 8 | 38.3% |
| 5 | 104 | 57 | 48 | 86 | 74 | 26 | 34.9% |
| 10 | 177 | 212 | 302 | 251 | 236 | 54 | 22.8% |
| 18 | 168 | 280 | 228 | 345 | 255 | 75 | 29.4% |
| 26 | 168 | 393 | 231 | 393 | 296 | 115 | 38.7% |
| 34 | 196 | 206 | 240 | 263 | 226 | 31 | 13.7% |
| 42 | 152 | 162 | 182 | 180 | 169 | 15 | 8.7% |
| 50 | 147 | 147 | 221 | 138 | 163 | 39 | 23.9% |

Formulation 4: 10% Hydroxytrienedione

| [h] | 1 | 2 | 3 | 4 | MW | SD | CV |
|---|---|---|---|---|---|---|---|
| 1 | 0 | 5 | 3 | 1 | 2 | 2 | 87.9% |
| 3 | 16 | 74 | 60 | 35 | 46 | 26 | 55.6% |
| 5 | 71 | 239 | 192 | 123 | 156 | 74 | 47.6% |
| 10 | 349 | 466 | 428 | 477 | 430 | 58 | 13.5% |
| 18 | 451 | 262 | 494 | 291 | 375 | 115 | 30.8% |
| 26 | 695 | 269 | 463 | 242 | 417 | 209 | 50.2% |
| 34 | 387 | 272 | 294 | 209 | 290 | 74 | 25.5% |
| 42 | 257 | 232 | 242 | 165 | 224 | 41 | 18.2% |
| 50 | 321 | 102 | 145 | 147 | 179 | 97 | 54.3% |

Formulation 5: 15% Hydroxytrienedione

| [h] | 1 | 2 | 4 | MW | SD | CV |
|---|---|---|---|---|---|---|
| 2 | 2 | 5 | 5 | 4 | 2 | 49.7% |
| 3 | 43 | 69 | 68 | 60 | 14 | 23.8% |
| 5 | 213 | 182 | 187 | 194 | 17 | 8.6% |
| 10 | 486 | 408 | 543 | 479 | 67 | 14.1% |
| 18 | 374 | 404 | 276 | 352 | 67 | 19.0% |
| 26 | 400 | 487 | 263 | 383 | 113 | 29.0% |
| 34 | 386 | 218 | 220 | 275 | 96 | 35.1% |
| 42 | 261 | 184 | 174 | 206 | 48 | 23.1% |
| 50 | 271 | 176 | 160 | 202 | 60 | 29.6% |

The mean values and standard deviations for the transdermal in-vitro-skin flows emerge from Table II below and are shown graphically in FIG. 2.

TABLE II
Mean Values and Standard Deviations:

| [h] | 1% | 3% | 5% | 10% | 15% |
|---|---|---|---|---|---|
| 1 | 1 ± 1 | 1 ± 0 | 1 ± 1 | 2 ± 2 | 4 ± 2 |
| 3 | 9 ± 6 | 16 ± 9 | 22 ± 8 | 46 ± 26 | 60 ± 14 |
| 5 | 32 ± 18 | 63 ± 38 | 74 ± 26 | 156 ± 74 | 194 ± 17 |
| 10 | 71 ± 28 | 151 ± 46 | 236 ± 54 | 430 ± 58 | 479 ± 67 |
| 18 | 66 ± 13 | 148 ± 22 | 255 ± 75 | 375 ± 115 | 352 ± 67 |
| 26 | 70 ± 31 | 152 ± 37 | 296 ± 115 | 417 ± 209 | 383 ± 113 |
| 34 | 47 ± 11 | 122 ± 39 | 226 ± 31 | 290 ± 74 | 275 ± 96 |
| 42 | 45 ± 15 | 101 ± 31 | 169 ± 15 | 224 ± 41 | 206 ± 48 |
| 50 | 35 ± 10 | 77 ± 25 | 163 ± 39 | 179 ± 97 | 202 ± 60 |

### Example 3

### Comparison Test Between Gestodene and Hydroxytrienedione

For comparison, the same measurements of the transdermal in-vitro skin flows—in otherwise identical formulation—were made with gestodene (GTD). The corresponding results emerge from the following tables.

Transdermal In-Vitro-Skin Flow in ng/cm²/h (1% by Weight-% of Gestodene)

| [h] | 1 | 2 | 3 | 4 | MW | SD | CV |
|---|---|---|---|---|---|---|---|
| 1 | 0 | 0 | 0 | 0 | 0 | 0 | — |
| 3 | 1 | 4 | 2 | 5 | 3 | 2 | 64.3% |
| 5 | 5 | 31 | 14 | 18 | 17 | 11 | 64.3% |
| 10 | 39 | 72 | 80 | 58 | 62 | 18 | 28.6% |
| 18 | 77 | 46 | 110 | 36 | 67 | 34 | 49.8% |
| 26 | 120 | 45 | 111 | 33 | 77 | 45 | 57.8% |
| 34 | 50 | 39 | 83 | 31 | 51 | 23 | 44.6% |
| 42 | 47 | 33 | 74 | 25 | 45 | 22 | 49.0% |
| 50 | 37 | 27 | 44 | 22 | 32 | 10 | 30.0% |

A matrix-TDS, in which the active ingredient is present in dissolved form, can be produced according to Example 1 with gestodene only up to about 1% (m/m). Moreover, it results in recrystallization phenomena of the gestodene. The skin flow cannot increase decisively.

The skin flows for 1%-matrix-TDS between hydroxytrienedione and gestodene are comparable, but allow the skin flows of hydroxytrienedione to increase significantly in that hydroxytrienedione in the matrix-TDS according to the invention is dissolved in a loadable manner up to about 20% (m/m).

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Matrix-transdermal system that contains (21S)-21-hydroxy-21-methyl-14,17-ethano-19-norpregna-4,9,15-triene-3,20-dione, characterized in that the matrix comprises polyacrylate adhesive.

2. Transdermal system according to claim 1, wherein the polyacrylate adhesive is a copolymer of at least two of the following monomers: 2-ethylhexylhexylacrylate, hydroxyethylhexylacrylate, vinyl acetate, vinyl pyrrolidone.

3. Transdermal system according to claim 2, wherein the polyacrylate adhesive is a copolymer that consists of 2-ethylhexylacrylate and hydroxyethylacrylate (Gelva^{(R)}) or a copolymer of these monomers with vinyl acetate and 2-ethylhexylacrylate-N-vinyl-2-pyrrolidone (TSR^{(R)}-adhesive of the Sekisui Company).

4. Transdermal system according to one of claims 1 to 3, characterized by a content of 0.1 to 20% by weight, preferably 1 to 15% of (21S)-21-hydroxy-21-methyl-14,17-ethano-19-norpregna-4,9,15-triene-3,20-dione in the matrix.

5. Transdermal system according to one of claims 1 to 4, wherein the matrix comprises at least one crystallization inhibitor.

6. Transdermal system according to claim 5, wherein the matrix comprises at least one N-vinyllactam polymer such as N-vinyl -1-aza-cycloheptan-2-one homopolymer, N-vinyl-piperidin-2-one homopolymer, polymers of vinylpyrrolidone such as polyvidone (Kollidon^{(R)}) or co-polymers of vinylpyrrolidone with vinyl acetate (copovidones).

7. Transdermal system according to one of claims 1 to 6, characterized by an additional content of at least one of the following penetration intensifiers:
Monovalent or multivalent alcohols such as ethanol, 1,2-propanediol or benzyl alcohol; saturated or unsaturated fatty alcohols with 8 to 18 carbon atoms, such as lauryl alcohol or cetyl alcohol; hydrocarbons such as mineral oil; saturated and unsaturated fatty acids with 8 to 18 carbon atoms such as stearic acid or oleic acid; fatty acid esters with up to 24 carbon atoms or dicarboxylic acid diesters with up to 24 carbon atoms, such as methyl ester, ethyl ester, isopropyl ester, butyl ester, sec-butyl ester, isobutyl ester, tert-butyl ester or monoglyceric acid ester of acetic acid, caproic acid, lauric acid, myristic acid, stearic acid and palmitic acid, phosphatide derivatives, such as lecithin, terpenes, urea and its derivatives or ethers, such as dimethyl isosorbide and diethylene glycol monoethyl ether.

8. Transdermal system according to claim 7, characterized by a content of at least one of the following penetration intensifiers:
lauryl alcohol, 1,2-propanediol, methyl ester and especially the isopropyl ester of myristic acid or oleic acid, diisopropyl adipate and diisopropyl sebacate, lauric acid and oleic acid, as well as mixtures thereof.

9. Transdermal system according to one of the preceding claims, characterized by an additional content of at least one estrogen such as estradiol, estriol, ethinylestradiol, derivatives thereof such as, for example, mono-esters and diesters such as estradiol-3,17β-diproprionate.

10. Transdermal system according to one of the preceding claims, characterized by an additional content of at least one stabilizer, such as, for example, β-cyclodextrin.
